(19) United States
(12) Patent Application Publication
(10) Pub. No.: US 2007/0021718 A1
(43) Pub. Date: Jan. 25, 2007

(54) Plastic spring

(76) Inventors: Stefan Burren, Bremgarten (CH); Ulrich Moser, Heimiswil (CH); Christian Schrul, Utzenstrof (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/439,522

(22) Filed: May 23, 2006

(30) Foreign Application Priority Data
Jul. 13, 2005 (DE) 10 2005 032 705.2
May 24, 2005 (DE) 10 2005 023 821.1

(51) Int. Cl. A61M 5/00

(52) U.S. Cl. 604/110; 604/263

(57) ABSTRACT

A spring for use as a compensating, supporting or driving spring of a device for a medical or pharmaceutical use, wherein the spring is molded from plastic by an injection molding process. The invention encompasses a device for a medical or pharmaceutical use, comprising a first component, a spring and a second component elastically supported against the first component by the spring.

### CROSS-REFERENCE TO RELATED APPLICATIONS

This application claims priority to German Application No. 10 2005 032 705.2, filed on Jul. 13, 2005 and German Application No. 10 2005 023 821.1, filed on May 24, 2005, the contents of both are incorporated in their entirety herein.

### BACKGROUND

The present invention relates to devices for dispensing, injecting or delivering substances, and to methods of making and using such devices. More particularly, the present invention relates to devices for medical or pharmaceutical applications or uses, and a spring for use in such devices. In some embodiments, the devices to which the invention relates may be used to obtain or draw a sample of body fluid.

Springs are used in a variety of functions in injection devices, infusion devices, administering devices or sampling devices, for example as a driving spring for a plunger or some other moveable element, or as a return spring for a plunger rod or another component of the device which can be moved against the return force of the relevant spring. In other functions, springs are used to support and fix or position one component of the device on or in another component, whereby the spring holds one of the components in a specific position relative to the other in a tensioned state, for example biases one of the components into an abutting contact with the other. This enables manufacturing and assembly tolerances to be compensated.

The components of injection devices, infusion devices, administering devices or sampling devices are typically molded parts made from plastic. Most are injection-molded parts. In basic injection devices, it is usually only the medicament container and the springs used in the device which are often not made of plastic. The medicament containers are usually glass ampoules, while the springs, in most cases, are coil springs made from steel. Most device manufacturers buy the springs from other sources. If they are produced on a mass scale, they are generally not suitable for the administering devices, which means that the construction of the device has to be adapted to the springs, which can only be obtained as mass-produced products. If a spring manufacturer makes the springs specially for a specific device, on the other hand, this drives up the price of the spring.

### SUMMARY

One object of the present invention is to provide a spring for use in a device for a medical or pharmaceutical use which can be more readily adapted to the specific nature of the device but is nevertheless inexpensive.

In some embodiments, a spring molded from plastic in an injection mold is used as a compensating, supporting or driving spring in a device for a medical or pharmaceutical application.

In some embodiments, the present invention comprises a device for a medical or pharmaceutical use, comprising a first component, a spring and a second component elastically supported against the first component by the spring.

In some embodiments, the present invention comprises a spring for use as a compensating, supporting or driving spring of a device for a medical or pharmaceutical use, wherein the spring is molded from plastic by an injection molding process.

The process of molding from plastic enables a spring to be produced so that the spring characteristic and design is optimally adapted to the function of the spring and the spatial requirements of the device with which it will be used. The components of the device on which the spring is supported or which are supported one on the other via the spring require less modification to these springs than is the case if using standard springs made from spring steel. In constructing the device, allowance no longer has to be made for the spring as a pre-defined component. It can be adapted to requirements to a much greater degree than in the past.

Devices for medical and pharmaceutical applications which are predominantly or to a large extent made from plastic components are very commonplace. Typical examples of such devices are injection devices, for example injection pens, for administering medicaments, usually in liquid form. A typical example of such devices are injection pens such as those used for the treatment of diabetes, administering growth hormones and in the treatment of osteoporosis, for example, and by means of which the respective user self-administers the medicament. Manufacturers of such devices usually make the individual components themselves, for example in the form of extruded parts or as injection molded parts, and assemble the device. Whereas the requisite springs were expensive parts which in the past had to be bought in, the springs proposed by the invention can now be produced by the same manufacturers themselves. Another factor which reduces cost is the fact that inexpensive plastic materials can be used.

In some embodiments, a spring in accordance with the present invention may be made from a composite plastic material. For example, reinforcing fibres may be incorporated in a matrix of a polymeric material. The fibre material may in turn be a metallic or optionally also ceramic material or may be made from another plastic different from the plastic matrix. In some preferred embodiments, the spring is made from a homogeneous plastic material, which may be a simple polymer, a copolymer or alternatively a blended material.

In one embodiment, a preferred plastic material is polyoxymethylene (POM). However, polyamides (Pa) are also suitable, for example. The E-modulus of the plastic material should be at least 1500 and not more than 4000 N/mm² as measured at the usual temperature of usage, i.e. the usual ambient temperatures.

From a structural point of view, it is of advantage to provide an integrally formed positioning device on an actual spring region, which will be referred to below as spring element, which can be simply and accurately adapted to the respective conditions at the assembly site. The purpose of the positioning device is to support the spring, i.e. either a resilient force to be generated by the spring is introduced into the spring by means of the positioning device if the spring is used exclusively as a compensating or supporting spring. Conversely, the spring transmits its spring force via the positioning device onto the relevant component of the device, wherein this approach is particularly crucial for use as a driving spring or return spring. By virtue of molding in the injection mold, the positioning device can be adapted, in regard to its form, to the specific design at the assembly site, depending on requirements, and can therefore replace the adapter pieces that are often necessary to transmit force. When using conventional steel springs, adapter pieces are often necessary as a means of making the force-transmitting surface of the spring larger or concentrating it on one or more specific points. Such functions can be fulfilled by the positioning device formed in a single piece with the spring region. In some preferred embodiments, the positioning device is molded in the form of a force-distributing ring or force-distributing part ring. A positioning device of this type is suitable for transmitting force from or onto a sleeve component to absorb or apply force from or to an end face, flange face or other shoulder face of the sleeve component as uniformly as possible.

The positioning device may be designed to assume more than just a pure supporting function, i.e. supporting the spring force, and can be improved to form a retaining mechanism, by which the spring is retained on one of the components between which the spring acts or on another component of the device. If the spring is retained on a third component, the function of the spring must not be detrimentally affected as a result, i.e. if retained in this manner, the spring will either be supported on a third component which moves jointly with one of the two other components or, although the spring is retained on the third component, it is displaceable relative to the third component. The means for retaining the spring may be a catch connection in particular, in which case the positioning device forms one of the catch elements for the catch connection to be produced or is formed in the already existing catch connection.

In addition to its actual spring function, the spring of another embodiment of the present invention also fulfils an extra function. In such an embodiment, the spring might serve as a guide, for example, for a component of the device mounted so as to be displaceable, e.g., a driving element or a dose metering element of the device, for example a plunger rod or a displaceable dose metering stop. The guide might be a linear guide, perhaps in combination with an anti-rotation lock. In another additional function or example, the spring may constitute a gear element, for example a threaded element, a driving device or dose metering mechanism of the device or, more generally expressed, a linking element, for example a rotating, screw or thrust joint. If the product is driven by means of a plunger and plunger rod, the plunger rod may be disposed in a threaded engagement with the spring, i.e. with a part of the spring fulfilling an additional function, and may be driven in a forward-drive direction in this threaded engagement in order to convey the product. Instead of a driving element or a dose metering element, the spring could be driven for conveying or metering purposes, for example driven forward in rotation.

A spring in accordance with the present invention may be a tension spring, flexion spring or torsion spring and, in some preferred embodiments, a compression spring. Its shape may be that of a coil spring, leaf spring, plate spring, a spring with legs, for example, or any suitable configuration. In many applications or uses it may be provided in the form of a coil spring. It may comprise a bellows, for example a folding bellows or a bellows with a meandering shape. In some preferred embodiments, the spring incorporates the spring element generating the elastic force and, if necessary, also several spring elements or components, and a positioning device of the type described above at one end of the spring element at least. In some embodiments, a positioning device may be provided at each end of the spring element, in which case the several positioning devices may each have an identical or a different mounted position or may be of an optimally adapted shape taking account of the respective function to be fulfilled.

The spring may serve as a driving spring of a mechanism for conveying a product which can be administered, for example insulin. It may also serve as a return spring for a needle guard of an injection pen, for example, or as a return spring for another component of the device, for example a return spring for a trigger button, and it may be used as a supporting or compensating spring. In the latter type of application, only a short spring path of about 1 to 3 mm is needed, for example. Accordingly, in such embodiments, the spring will have a spring path of at most about 5 mm or, in some preferred embodiments, at most about 3 mm, for example. In the case of a coil spring or a spring bellows, this means that it or they will have a maximum spring path of about 5 mm, or about 3 mm, for example, from the fully relaxed state into the state in which the spring coil or coils or bellows elements lie on a block. Any suitable relaxed or tensed length may be used. In one application as a compensating spring, for example, the spring may serve as a means of compensating longitudinal tolerances for supporting a medicament container in an injection device, such as an injection pen.

### BRIEF DESCRIPTION OF THE DRAWINGS

FIG. 1 shows a distal portion of an injection device with a first embodiment of a spring in accordance with the present invention, which spring serves as a compensating spring providing support for a medicament container,

FIG. 2 shows a first view of the spring,

FIG. 3 shows a second view of the spring, and

FIG. 4 shows a distal portion of an injection device with a second embodiment of a spring in accordance with the present invention.

### DETAILED DESCRIPTION

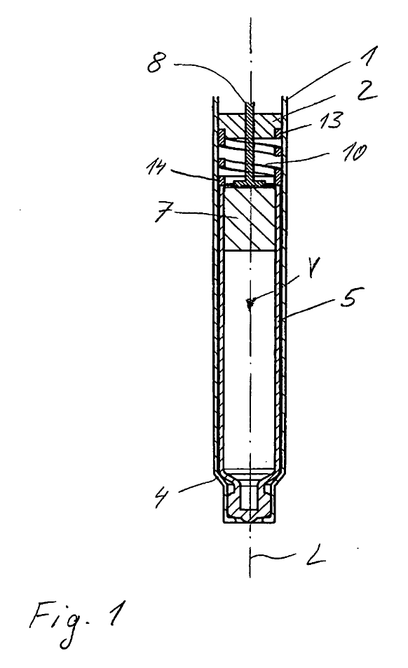

FIG. 1 illustrates a distal portion (or front end portion) of an injection device, which in the embodiment illustrated as an example is an injection pen, wherein the device has a central longitudinal axis L. The injection device has a longitudinally extending, sleeve-type housing 1 comprising one or more parts. Accommodated in a distal portion of the housing 1 is a container 5, for example a glass ampoule, which is filled with a liquid medicament, for example insulin. At a distal end, the container 5 has an outlet. A driving element 7 in the form of a plunger is accommodated in the container 5. The driving element 7 closes off the container 5 in a liquid-tight seal. When the driving element 7 is moved in a forward drive direction V towards the outlet, the product or, if the device incorporates setting mechanism or means, a set dose of the product, is forced out of the container 5 and administered. In addition to the driving element 7, a driving mechanism has another driving member 8, which is a plunger rod in the embodiment illustrated as an example. Only a distal portion of the driving member 8 is shown in the embodiment illustrated as an example. In one preferred embodiment, the injection device is also equipped with a dose metering mechanism, which is accommodated in a proximal portion (or rear end portion) of the housing 1, not illustrated. Also disposed in the housing 1 is an insert 2, which is secured so that it can not move axially. The plunger rod 8 extends through the insert 2. The insert 2 guides the plunger rod 8 axially in a linear movement. The insert 2 may preferably also joined to the housing 1 so that it is prevented from rotating and forms an anti-rotation lock for the driving member 8. The insert 2 lies axially opposite a proximal end of the container 5.

In the forward drive direction V parallel with the longitudinal axis L of the driving element 7, the container 5 is supported by means of an abutment contact on the housing 1. In the embodiment illustrated as an example, the housing 1 itself directly forms a stop 4 for the container 5. In principle, however, a stop 4 may be provided in the form of a part joined to or carried by or in the housing 1 so that it can not move axially. In the direction opposite the forward drive direction V, the container 5 is supported on the housing 1 by means of a spring 10 and the insert 2. The spring 10 is provided in the form of a coil spring and operates as a compression spring. The longitudinal axis L of the device also constitutes the spring axis. The spring 10 bridges a space left free between the container 5 and the insert 2 and biases the container 5 against the stop 4 in the forward drive direction V. In the embodiment illustrated as an example, the spring 10 is directly supported on the container 5 and is so in the embodiment illustrated as an example against its terminal-face proximal end. For supporting the spring 10 the insert 2 forms a shoulder surface pointing in the direction opposite the forward drive direction V, from which a flat base surrounded by the spring 10 projects in the forward drive direction V. The spring 10 can be connected to the insert 2, in particular in a friction contact, by means of the base and retained on the insert 2 thereby. Other suitable connection methods or structures may be used.

In the direction of the longitudinal axis L of its spring axis, the spring 10 has a spring portion 11 and a positioning device 13, 14 at the two terminal-face ends of the spring portion 11 respectively. The spring portion 11 forms a coil spring with a single spring turn, which merges into the positioning device 13 at one axial end and into the positioning device 14 at the other axial end. The positioning devices 13, 14 are each provided in the form of flat rings, with flat end faces 13a, 14a disposed at a right angle to the longitudinal axis L at their axial end faces. The end faces 13a, 14a constitute support surfaces, by means of which the spring 10 is supported on the container 5 and the insert 2 solely by contact pressure.

The spring 10, in one embodiment comprising the spring portion 1 1 and the positioning devices 13, 14, is homogeneously molded in a single piece from a polymer material by an injection molding process. The polymer material is POM but as an alternative could also be PA or other suitable material, for example.

The spring 10 is of a hollow circular cylindrical shape and has a constant diameter across its entire length. Across its axial length, the spring portion 11 likewise has a substantially constant internal diameter. The wall thickness of the spring portion 11 is between about 0.5 and 2 mm. The positioning devices 13, 14 are identical. Their circular cylindrical internal cross-section is smaller than the circular cylindrical internal cross-section of the spring portion 11. Accordingly, the support surfaces 13a, 14a obtained are wider than the spring portion 11.

The spring portion 11 turns about the longitudinal axis L by virtually 360°. Overall, it has a width B as measured in the direction of the longitudinal axis L. The width B is bigger, in some embodiments significantly larger, than the thickness D as measured in the radial direction and, in the embodiment illustrated as an example, the width B is approximately five times as big as the thickness D of the spring portion 11.

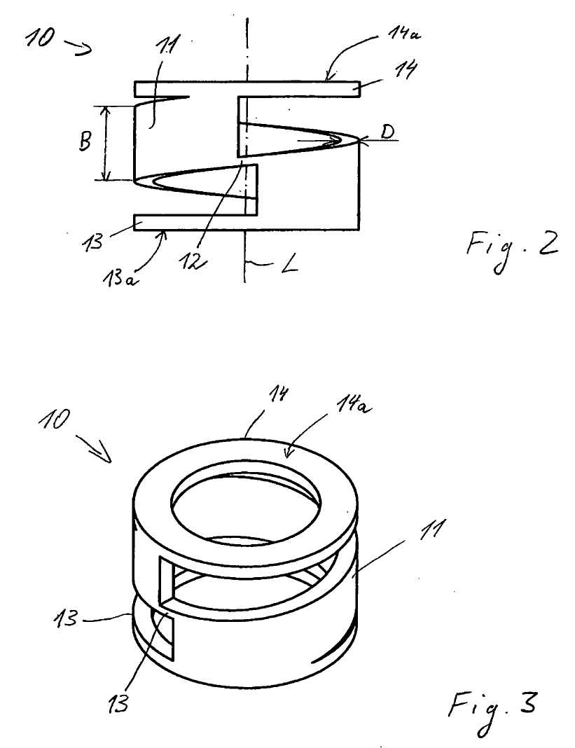

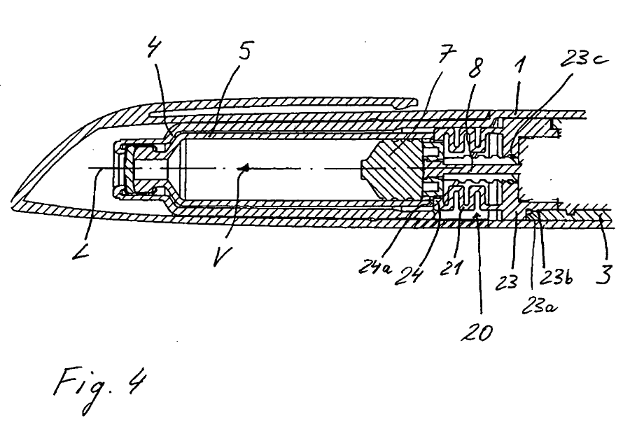

FIGS. 2 and 3 illustrate the spring 10 in the state of its original shape before its initial use. As viewed in the circumferential direction, the spring portion 11 has two ends, which axially overlap with one another by a width approximately corresponding to the thickness D. A distance left free between the ends as measured in the circumferential direction is bridged by a connecting web 12. The width B and the pitch of the spring turn are selected so that this slight axial overlap is obtained. The connecting web 12 has the same pitch as the spring turn and extends the end face of the spring turn facing the positioning device 13 at one axial end and extends the end face of the spring turn facing the positioning device 14 at the other axial end with respect to their respective tangential ends. The two tangential ends of the spring turn extend axially parallel.

When pressure is applied to the spring 10, the connecting web 12 breaks and the two tangential ends of the spring portion push past one another axially to an increasing degree. However, a narrow axial slit remains free between the two ends. If the injection device is a device which is disposed of once the container 5 has been emptied, the clicking noise which occurs as the container 5 is inserted may be used by the manufacturer as an indicator that it has been correctly inserted because the occurrence of the clicking noise guarantees that the spring has pushed the container 5 against the stop 4 by means of its spring force. If the device is one which can be repeatedly re-used, the same applies during a change of container, when the spring 10 is also replaced.

As may be seen in particular from FIG. 3, the positioning device 14 by means of which the spring 10 is pushed against the container 5 constitutes a support surface 14a enlarged in the radial direction. The positioning device 14 therefore fulfils the function of a base disc, which distributes the spring force uniformly on the proximal edge of the container 5. This also ensures that the spring 10 acts circumferentially on the container 5 and is also supported circumferentially on the insert 2. This helps prevents any tilting forces. The external surface of the spring portion 11 and also the external surfaces of the positioning devices 13, 14 are selected so that the spring 10 has a narrow sliding contact with the internal surface of the housing I and the housing 1 axially guides the spring 10 as it springs in and out.

FIG. 4 illustrates another exemplary embodiment of the present invention, in this case, a distal portion of an injection device which, in the embodiment illustrated as an example, is again an injection pen, with a second embodiment of a spring 20. Components which fulfil the same function are denoted by the same reference numbers as those used for the embodiment illustrated as a first example, and reference may therefore be made to the explanations given in connection with it. The housing of the embodiment illustrated as a second example has a distal housing portion 1 accommodating the container 5 and a proximal portion 3 which is connected to the housing portion 1 so that it can not be moved axially and which is preferably also prevented from rotating. The housing portion 3 supports members of a dose metering mechanism as well as members of the driving mechanism of the injection device.

The spring 20 again serves as a compensating spring for the container 5. In the forward drive direction V, it is supported on the proximal end of the container 5. In the direction opposite the forward drive direction V, it is supported by means of a support surface 23a on the proximal housing portion 3. The spring 20 comprises a proximal positioning device 23, a distal positioning device 24 and a spring element 21 disposed between the positioning devices 23 and 24. In longitudinal section, the spring element 21 is of a meandering shape with several portions extending alternately in the forward drive direction V and transversely thereto. This results in a spring bellows, the spring action of which can be likened to that of flexible arms connected in series. A compensating spring 20 is obtained as a result, by means of which the container 5 is elastically supported on the housing 1, 3 in and opposite the forward drive direction V. The spring element 21 may, although it does not necessarily have to, form a closed casing about the longitudinal axis L.

The distal positioning device 24 is in loose contact with the container 5, i.e. there exists only a pressing contact with the container 5. In terms of function, the positioning device 24 generally corresponds to the positioning device 14 described in connection with the embodiment given as a first example. Not only does the positioning device 23 serve as an axial support, namely by means of its support surface 23a pointing in the distal direction, it also assumes the function of a retaining mechanism 23b by means of which the spring 20 is held in a latched or snap-fit connection with the proximal housing portion 3. In the embodiment illustrated as an example, an outwardly projecting cam formed on an external face of the positioning device 23 constitutes the retaining mechanism 23b. Accordingly, the catch complementary element of the housing portion 3 is a recess and, in the embodiment illustrated as an example, is a recess in an internal face of the housing portion 3. When the injection device is in the assembled state, the cam constituting the retaining mechanism 23b latches in the recess of the housing portion 3. As a result, the spring 20 is retained on the housing portion 3 on the one hand and is disposed so that it can not rotate relative to the housing portion 3, on the other hand, i.e. is retained so that it is prevented from rotating.

In the region of the positioning device 23, the spring 21 additionally constitutes a linking element 23c of a link, the complementary linking element of which constitutes a plunger rod 8, which acts on the plunger 7 in the forward drive direction V. The spring 21 and the plunger rod 8 form a screw link with one another. As a linking element 23c, the spring 21 acts as a threaded nut. The plunger rod 8 is the link of complementary element forming a threaded rod. When the plunger rod 8 is driven in rotation, the resultant link is moved in the forward drive direction V.

Embodiments of the present invention, including preferred embodiments, have been presented for the purpose of illustration and description. They are not intended to be exhaustive or to limit the invention to the precise forms and steps disclosed. Obvious modifications or variations are possible in light of the above teachings. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A spring for use as one of a compensating, supporting or driving spring of a device for a medical or pharmaceutical use, said spring molded from plastic by an injection molding process.

2. The spring as claimed in claim 1, wherein the spring is made exclusively from plastic.

3. The spring as claimed in claim 1, wherein the spring comprises a spring element comprising an elastic bellows.

4. The spring as claimed in claim 1, wherein the spring comprises a spring element comprising a coil spring with at least one spring turn.

5. The spring as claimed in claim 4, wherein in a tensed state, the at least one spring turn has two ends with a slit between them and, after molding in the injection mold, said ends are joined to one another across the slit by a web which is broken after molding.

6. The spring as claimed in claim 4, wherein the spring turn has two mutually facing ends and a connecting web connecting the ends to one another and is able to break when tension is applied to the spring.

7. The spring as claimed in claim 1, wherein the spring comprises a spring element and a positioning device molded jointly in the injection mold or injected onto one another in the injection mold, said positioning device applying pressure to the spring element by a tensioning force.

8. The spring as claimed in claim 7, wherein the positioning device comprises at least one support surface via which a tensioning force is applied to the spring.

9. The spring as claimed in claim 8, wherein tension is applied to the spring along a spring axis by applying pressure, and the at least one support surface extends about the spring axis across at least a major part of a full circumference.

10. The spring as claimed in claim 1, wherein the device comprises another component and wherein the spring comprises a linking element for forming a link with the another component.

11. A device for a medical or pharmaceutical use, said device comprising:
a) a first component;
b) a spring as molded from a plastic; and
c) a second component elastically supported on the first component by the spring.

12. The device as claimed in claim 1 1, wherein the device is one of a device for administering a fluid product or a device for drawing a sample of a body fluid.

13. The device as claimed in claim 12, the second component comprises a container for the product or body fluid, the first component comprises a stop, and the spring pushes the container against the stop.

14. The device as claimed in claim 11, wherein the spring comprises a spring element and a positioning device molded jointly in an injection mold or injected onto one another in the injection mold, said positioning device fixing the spring element in one of translation or rotation on one of the at least one of the components or another component of the device.

15. The device as claimed in claim 11, wherein the first component comprises a portion of a housing accommodating the second component.

16. The device as claimed in claim 15, wherein the first component, comprises a stop for the second component and comprises one of an insert or another housing portion on which the spring is supported and which is connected to the housing to be non-displaceable in the direction in which the spring acts.

17. The device as claimed in claim 16, wherein the portion of the housing accommodating the second component, and the insert or other housing portion, are releasably connected to one another to enable the second component to be replaced.

18. The device as claimed in claim 15, wherein the portion of the housing accommodating the second component, and an insert or an other housing portion are non-releasably connected to one another or are a single piece.

19. The device as claimed in claim 11, wherein the spring comprises a retaining mechanism by which it is retained on one of the components.

20. The device as claimed in claim 19, wherein the retaining mechanism comprises a catch connection.

21. The device as claimed in claim 11, wherein the device further comprises a driving member or a dose metering member, and the spring and the driving member or dose metering member form a link in conjunction with one another.

22. The device according to claim 21, wherein the link is one of a screw link or a thrust link.

23. The device as claimed in claim 11, wherein the spring is connected to one of the first or second component such that it is prevented from rotating.
